(19) United States
(12) Patent Application Publication
(10) Pub. No.: US 2021/0059394 A1
(43) Pub. Date: Mar. 4, 2021

(54) ORAL HEALTH DENTAL PRODUCT DISPENSING TOOTHBRUSH AND ORAL HEALTH SYSTEM INCLUDING TOOTHBRUSH AND DENTAL PRODUCT

(71) Applicant: ALBERT RUIZ, Sebring, FL (US)

(72) Inventor: ALBERT RUIZ, Sebring, FL (US)

(21) Appl. No.: 17/010,255

(22) Filed: Sep. 2, 2020

Related U.S. Application Data
(60) Provisional application No. 62/895,255, filed on Sep. 3, 2019.

(51) Int. Cl. A46B 11/00; A61C 17/02; A46B 9/04; A46B 5/00; A46B 17/06; A61L 2/18

(52) U.S. Cl. CPC A46B 11/0041; A61C 17/0202; A46B 9/04; A46B 11/0058; A61L 2202/15; A46B 17/065; A61L 2/18; A46B 2200/1066; A46B 5/0095

(57) ABSTRACT

In one aspect, the present invention relates to an oral health dental product dispensing toothbrush configured to selectively spray an oral health dental product directly into a user's mouth during use of the toothbrush and configured to selectively spray the oral health dental product directly onto bristles of a brush head in order to disinfect and/or sanitize the bristles. The oral health dental product is sprayed into the user's mouth and onto the brush bristles in a controlled manner and in the form of a fine, gentle, dispersed mist. In another aspect, the present invention relates to an oral health system including one or more of the toothbrushes according to the first aspect and one or more oral health dental products for use with the one or more toothbrushes.

### RELATED APPLICATION

This application claims the benefit of U.S. Provisional Patent Application No. 62/895,255, filed on Sep. 3, 2019.

### BACKGROUND

### Field

The present invention relates generally to oral health and hygiene, and more particularly to an oral health dental product dispensing toothbrush suitable for promoting and improving good dental hygiene during brushing operations and for preventing oral disease. The oral health dental product dispensing toothbrush of the present invention is also configured to maintain a brush portion of the toothbrush in a cleaned and sanitized state between uses of the toothbrush. The present invention also relates to an oral health system containing at least one toothbrush of the present invention and at least one oral health dental product for use with the toothbrush.

### Background Information

The advantages of good dental hygiene are well known, particularly for the prevention of oral diseases. In addition to frequent brushing of teeth, many medicated and non-medicated dental products currently existing in the market have been used to maintain oral health and hygiene, including dental rinses, mouth sprays, anti-cavity oral rinses, mouth cavity cleansers and mouth sprays. However, since such dental products are not associated with a particular toothbrush and requires applications separate from the toothbrush, users frequently either forget to use or intentionally forego using such dental products. Furthermore, even when such dental products are used, it has been difficult to apply such dental products in a controlled manner so as to maximize the effectiveness of the dental products.

Moreover, it is well-known that toothbrushes attract harmful microorganisms such as bacteria, viruses and microbes. Medical studies have indicated that unsterile toothbrushes are contributing factors to sore throats, bacterial and viral infections and periodontal disease. Accordingly, maintaining toothbrushes in a cleaned or sanitized state, particularly between uses of the toothbrush, has been a paramount concern.

In many instances, consumers have sought to mitigate this problem by frequently replacing their toothbrush, and sometimes after only a few uses. This has resulted in increased costs, particularly for more expensive, high-end toothbrushes. Nevertheless, new toothbrushes have been found to contain bacteria on their bristles, such that the frequent replacement of a toothbrush may not obviate the problem of bacterial contamination or the spread of disease to the user.

Many attempts have been made to clean the brush portion of toothbrushes by spraying a cleansing medium onto the brush portion from the outside of the toothbrush. However, this has proved to be unpleasant both for the user and the environment as a result of the cleansing medium being fed to the brush head in an uncontrolled way. Other previously known solutions for maintaining toothbrushes in a cleaned and sanitized state have been proven to consist of costly structure and be complicated, including lacking user-friendliness and reliability.

In view of the foregoing, there is a need for an improved toothbrush which overcomes the drawbacks of the conventional art, including the above-mentioned inconveniences and/or limitations.

### SUMMARY

It is an object of the present disclosure to provide an oral health dental product dispensing toothbrush suitable to promote and improve good dental hygiene during brushing operations and prevent oral disease.

Another object of the present invention is to provide an oral health dental product dispensing toothbrush configured to maintain the brush portion of the toothbrush in a cleaned or sanitized state between uses of the toothbrush.

Another object of the present is invention is to provide an oral health dental product dispensing toothbrush configured to spray an oral health dental product in a controlled manner directly into a user's mouth during use of the toothbrush. In an embodiment, the oral health dental product is sprayed in the form of a tine, gentle, dispersed mist.

A further object of the present invention is to provide an oral health dental product dispensing toothbrush configured to spray an oral health dental product in a controlled manner directly onto bristles of the toothbrush in order to decontaminate and/or sanitize the toothbrush bristles. According to a feature of the present invention, the oral health dental product is sprayed onto the bristles in the form of a fine, gentle, dispersed mist.

A further object of the present invention is to provide a self-sanitizing spray toothbrush having dual functionalities, namely, for operation by a user while brushing the teeth to selectively dispense an oral health dental product directly into the user's mouth to promote and improve good dental hygiene, and for operation by a user when a brush head of the toothbrush is covered with a cap to selectively dispense an oral health dental product directly onto bristles of the brush head for disinfecting or decontaminating the bristles. According to a feature of the present invention, the oral health dental product is dispensed in the form of a fine, gentle, dispersed mist.

Still another object of the present invention is to provide an oral health dental product dispensing toothbrush as described herein which is reliable and easy to use by consumers.

Yet another object of the present invention is to provide an oral health system containing one or more toothbrushes and/or toothbrush components according to the present invention and one or more dental products for use with the toothbrush.

A preferred embodiment of one aspect of the invention provides an oral health dental product dispensing toothbrush comprising a grip portion including a reservoir for an oral health dental product, a brush portion and a cap for covering the brush portion. The reservoir is connected with the brush portion by means of a pump device in order to spray the oral health dental product in a controlled manner directly into a user's mouth during use of the toothbrush and to spray the oral health dental product in a controlled manner directly onto bristles of the brush portion when the brush portion is covered with the cap. According to a feature of the present invention, the oral health dental product is sprayed in the form of a fine, gentle, dispersed mist.

A preferred embodiment of another aspect of the invention provides an oral health system containing one or more toothbrushes and/or toothbrush components according to the present invention and one or more dental products for use with the toothbrush. In one example of this embodiment, the toothbrush components include a preselected number of removable/replaceable toothbrush heads, and the dental products include a fluid (e.g., a liquid) including one or a combination of a dental rinse, mouth spray, anti-cavity oral rinse, mouth cavity cleanser, mouth spray, dentifrice, freshener, and antibacterial/antiviral for therapeutic, oral health-hygiene-cosmetics, or in any otherwise application.

Other objects, features and advantages of the invention will be apparent with the following detailed description taken in conjunction with the accompanying drawings showing preferred embodiments of the invention.

### BRIEF DESCRIPTION OF THE DRAWINGS

The foregoing summary, as well as the following detailed description of preferred embodiments of the disclosure, will be better understood when read in conjunction with the accompanying drawings. For the purpose of illustrating the disclosure, there is shown in the drawings embodiments which are presently preferred. It should be understood, however, that the disclosure is not limited to the precise arrangement and instrumentalities shown.

FIG. 1 is a front elevational view of an oral health dental product dispensing toothbrush according to an embodiment of the present invention;

FIG. 2 is a cross-sectional view of the oral health dental product taken along cross-sectional line A-A in FIG. 1;

FIG. 3 is a right-side elevational view of the oral health dental product dispensing toothbrush shown in FIG. 1;

FIG. 4 is a top view of the oral health dental product dispensing toothbrush shown in FIG. 1;

FIG. 5 is a bottom view of the oral health dental product dispensing toothbrush shown in FIG. 1;

FIG. 6 is a left-side perspective view of the oral health dental product dispensing toothbrush shown in FIG. 1;

FIG. 7 is a view similar to FIG. 6, but with the brush head cap removed;

FIG. 8 is an exploded view of the oral health dental product dispensing toothbrush shown in FIG. 1;

FIG. 9 is a front elevational view of an oral health dental product dispensing toothbrush according to another embodiment of the present invention;

FIG. 10 is a rear elevational view of the oral health dental product dispensing toothbrush shown in FIG. 9;

FIG. 11 is a cross-sectional view of the oral health dental product taken along cross-sectional line B-B in FIG. 10;

FIG. 12 is a left-side elevational view of the oral health dental product dispensing toothbrush shown in FIG. 9;

FIG. 13 is a right-side elevational view of the oral health dental product dispensing toothbrush shown in FIG. 9;

FIG. 14 is a left-side perspective view of the oral health dental product dispensing toothbrush shown in FIG. 9;

FIG. 15 is a right-side perspective view of the oral health dental product dispensing toothbrush shown in FIG. 9; and

FIG. 16 is an exploded view of the oral health dental product dispensing toothbrush shown in FIG. 9.

### DETAILED DESCRIPTION

The present disclosure now will be described more fully hereinafter with reference to the accompanying drawings, in which exemplary embodiments of the disclosure are shown. This disclosure may, however, be embodied in many different forms and should not be construed as limited to the embodiments set forth herein; rather, these embodiments are provided so that this disclosure will be thorough and complete, and will fully convey the scope of the disclosure to those skilled in the art.

For convenience of description, the terms “front”, “back”, “upper”, “lower”, “top”, “bottom”, “front”, “rear”, “right”, “left”, “side” and words of similar import will have reference to the various members and components of the oral health dental product dispensing toothbrush of the present disclosure as arranged and illustrated in the figures of the drawings and described hereinafter in detail.

It should also be understood that the terms “about,” “approximately,” “generally,” “substantially” and like terms, which may be used herein when referring to a dimension or characteristic of a component of the present disclosure, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

As described in this application, “oral health dental product” (also referred to herein as “dental product”) refers to a fluid (e.g., liquid), either medicated or non-medicated, including one or a combination of a dental rinse, mouth spray, anti-cavity oral rinse, mouth cavity cleanser, mouth spray, dentifrice, freshener, and antibacterial/antiviral for therapeutic, oral health-hygiene-cosmetics, or in any otherwise application. Additionally, as described in this application, “spray”, “spraying” or “sprayed” refers to a fine, gentle, dispersed mist of a fluid, such as the projection of a liquid in a dispersive manner.

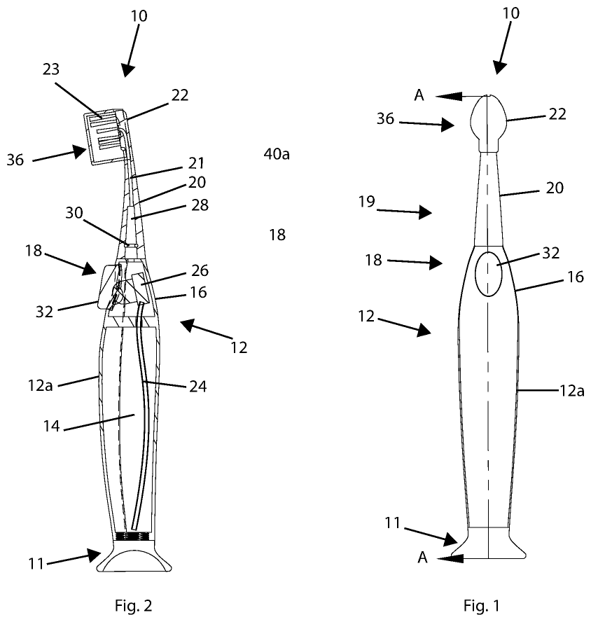

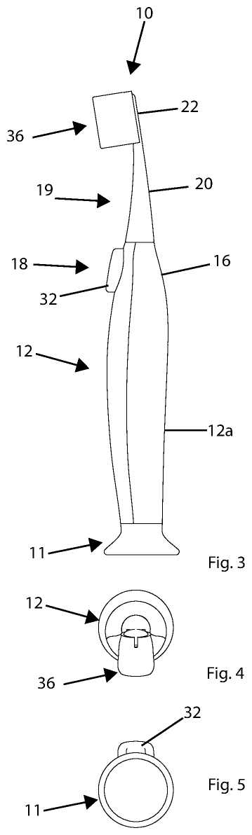

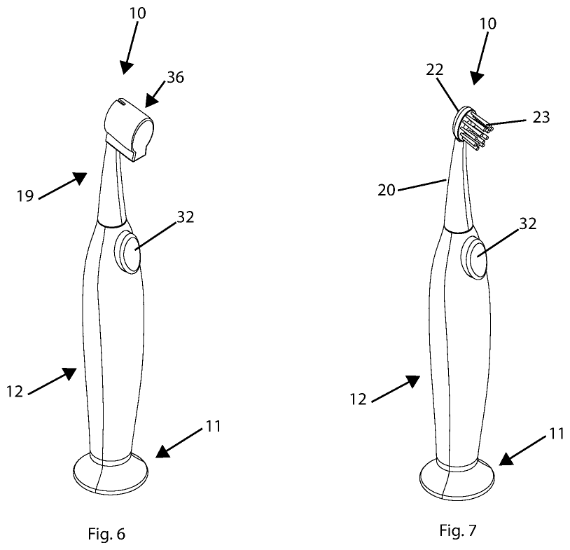

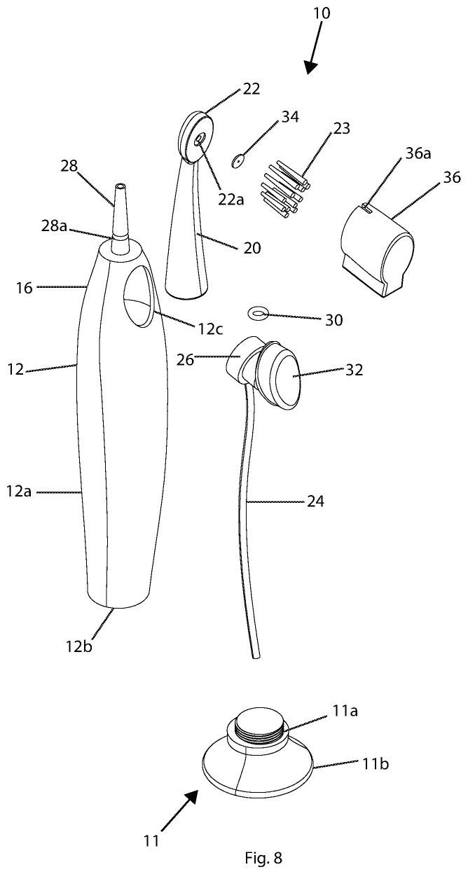

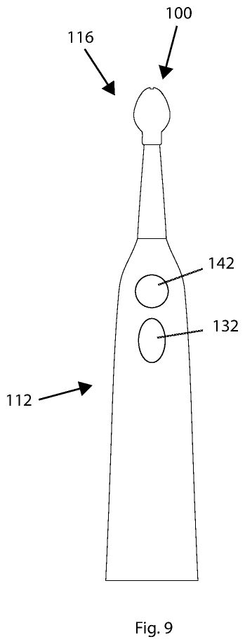

FIGS. 1-8 illustrate one exemplary embodiment of the oral health dental product dispensing toothbrush, generally designated with the arrow identified with reference numeral 10, according to the present invention. In this embodiment, toothbrush 10 is configured as a manual toothbrush and comprises components generally designated with arrows identified with the following reference numerals: a removable end cap 11; a body 12; a manual pump mechanism 18; a removable/replaceable a neck/head assembly 19; and a removable brush cap or cover 36.

Body 12 includes a grip portion 12a and a frame portion 16 defining an interior space forming an integrated, refillable container portion or reservoir 14 configured to be filled with and contain an oral health dental product. Grip portion 12a extends from a first side of frame portion 16. Neck/head assembly 19 includes an arm portion 20 and a brush portion or head 22 provided with bristles 23. Arm portion 20 is formed in one piece with brush portion 22 and extends from a second side of frame portion 16 opposite the first side thereof.

Reservoir 14 is contained within body 12 and has an aperture 12b for filling reservoir 14 with an oral health dental product. End cap 11 is configured for removable connection to and disconnection from a lower open end 12b of body 12 to permit an oral health dental product to be filled into reservoir 14 as well as to be removed therefrom as desired. In this embodiment, end cap includes a male thread portion 11a and a suction cup 11b. Male thread portion 11a is configured for threaded engagement with a female thread portion formed in an interior surface at lower open end of body 12, and suction cup 11 is positioned and configured for firmly supporting toothbrush 12 on a surface, such as during periods of non-use of toothbrush 10.

A pipe or tube 24 extends into reservoir 14 and is connected to a pump element 26 of pump mechanism 18. An attachment nozzle 28 having an exterior conical surface is integrally connected to and extends from the second side of frame portion 16 and is configured to be inserted into conduit 21 inside arm portion 20. A lower end of attachment nozzle 28 is configured for connection in fluid communication with pump element 26. Arm portion 20 is configured to receive and be removably attached to nozzle 28 via pressure fit by means of the exterior conical surface of nozzle 28 engaging a corresponding generally conically-shaped inner portion of conduit 21 in the manner shown in FIG. 2. The exterior conical surface of nozzle 28 is provided with a groove 28a configured to receive an O-ring 30 so that a fluid-tight connection is achieved between arm portion 20 and nozzle 28. By this construction, nozzle 28, and thus neck/head assembly 19, is in fluid communication with pump mechanism 20 and the interior of reservoir 14.

Manual pump mechanism 18 includes an activating member in the form of a push-button 32 associated with pump element 26 by means of which a pressure or suction effect is obtained in order to transfer the oral health dental product from reservoir 14 to brush portion 22. In the assembled state of toothbrush 10, push-button 32 is exposed from an aperture 12c of body 12 and accessible to the user for actuating manual pump mechanism 18. Pump mechanisms for toothbrushes are well-known in the art. Any known pump mechanism which can effectively transfer the oral health dental product from reservoir 14 to brush portion 22 is suitable and can be used for toothbrush 10 without departing from the spirit and scope of the present invention.

Neck/head assembly 19 can be removed from body 12 by disconnecting arm portion 20 from nozzle 28. Toothbrush 10 according to the present invention is therefore configured for use with various interchangeable neck/head assemblies (i.e., brush heads) to provide the user with a total cleaning experience. Brush portion 22 is formed with an orifice 22a in fluid communication with conduit 21. A spray nozzle 34 is provided in association with orifice 22a at a location corresponding generally to the root of bristles 23.

During use of toothbrush 10 (e.g, a brushing operation) and upon actuation of manual pump 18 via push-button 32 (e.g., by pressing push-button 32 downward) such that the oral health dental product is transferred from reservoir 14 to brush portion 22, spray nozzle 34 is configured to spray the oral health dental product in a controlled manner directly into a user's mouth. Preferably, spray nozzle 34 is configured to spray the oral health dental product directly into the user's mouth to effectively cover major areas inside the user's mouth. This configuration and use of toothbrush 10 promote and improve good dental hygiene and prevent oral disease by allowing users to clean the teeth properly and to provide therapeutic and freshening dental product applications, for example. As described above, the toothbrush 10 is configured to spray the oral health dental product in the form of a fine, gentle, dispersed mist.

Cap 36 is configured (e.g., preferably congruent with the shape of brush portion 22) to completely cover and seal bristles 23 of brush portion 22, as shown in the figures, for substantially preventing bristles 23 from any kind of contamination from external sources, as well as for decontaminating bristles 23 after each use of toothbrush 10 as further described below. Cap 36 includes a vent aperture 36a for air circulation, including for drying bristles 23 after each use. Cap 36 therefore constitutes a hygienic cap for toothbrush 10.

According to the present invention, when brush portion 22 is covered and sealed with cap 36, upon actuation of manual pump mechanism 18 via push-button 32 such that the oral health dental product is transferred from reservoir 14 to brush portion 22, spray nozzle 34 is configured to spray the oral health dental product in a controlled manner directly onto bristles 23 of toothbrush 10 in order to disinfect (e.g., decontaminate, sanitize) toothbrush bristles 23. Due to the sealing function of cap 36 relative to brush portion 22, the oral health dental product remains within cap 36 after being sprayed therein as described above to enhance the disinfecting function. By disinfecting bristles 23 in this manner while brush portion 22 is covered with cap 36, toothbrush 10 according to the present invention has the potential to substantially reduce the chances of re-introducing bacteria to the mouth that may otherwise linger on toothbrush 10, particularly when the user is suffering from an illness.

By the foregoing construction and corresponding functions, toothbrush 10 according to the present invention constitutes a self-sanitizing spray toothbrush having dual operational functionalities, namely, for operation by a user while brushing the teeth to selectively dispense an oral health dental product directly into the user's mouth to promote and improve good dental hygiene, and for operation by a user when brush head 22 is covered with cap 36 to selectively dispense an oral health dental product directly onto bristles 23 for disinfecting or decontaminating the bristles 23. The self-sanitizing spray toothbrush according to the present invention provides a convenient method for the general improvement of oral hygiene practices, including substantially reducing the risk of cross-infection between a sick family member and those who store their toothbrushes within close proximity of the infected toothbrush.

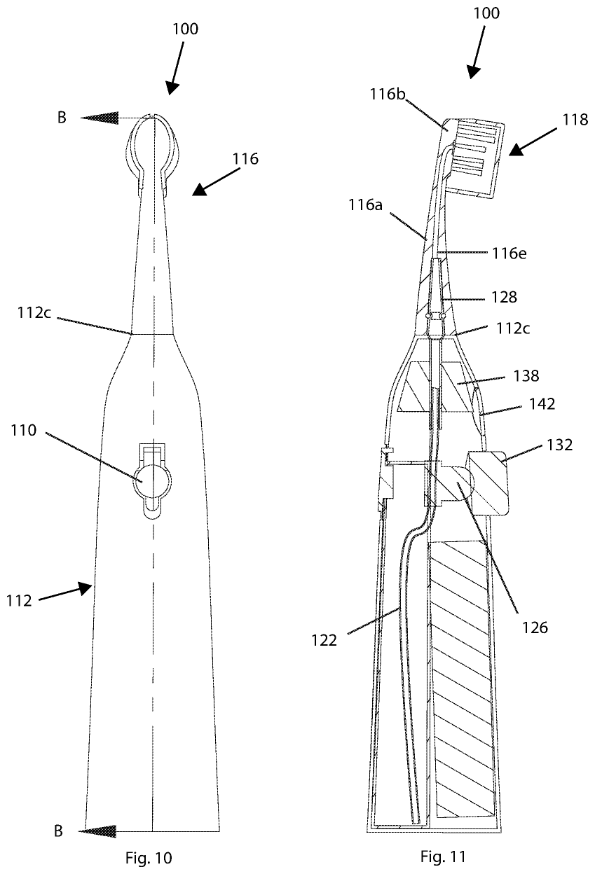

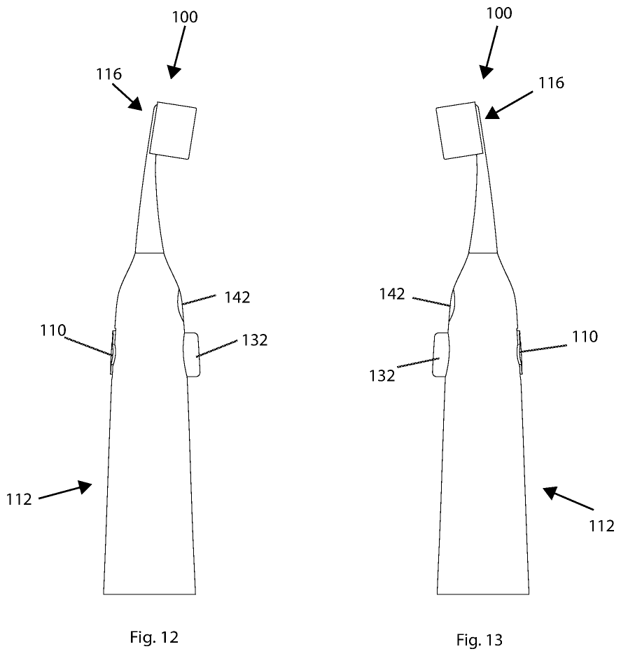

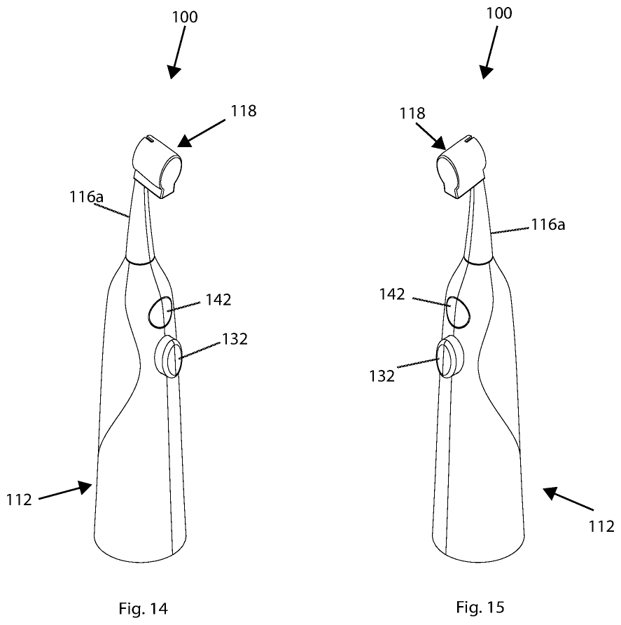

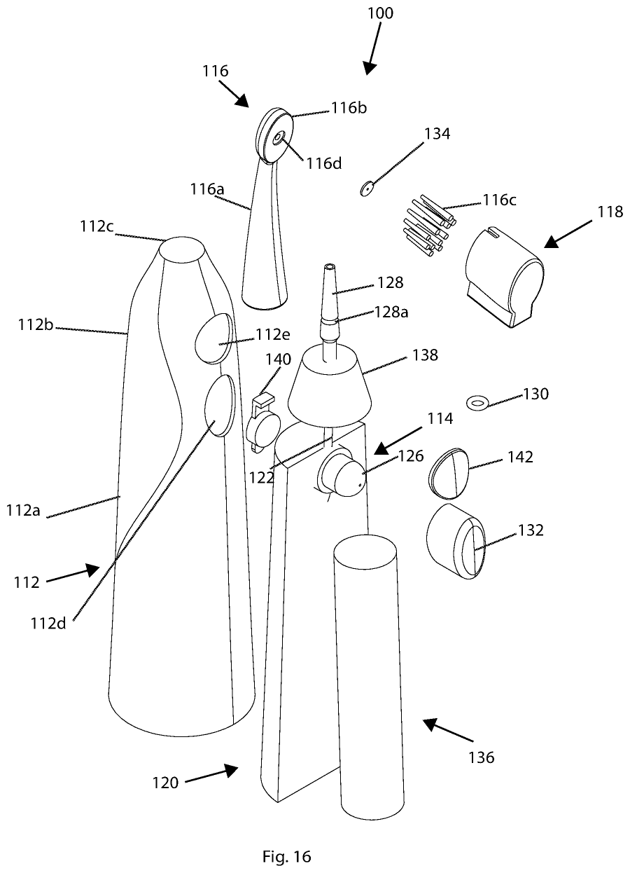

FIGS. 9-16 illustrate an oral health dental product dispensing toothbrush, generally designated at 100, according to another exemplary embodiment of the present invention. In this embodiment, toothbrush 100 is configured as an electric toothbrush and comprises components generally designated with arrows identified with the following reference numerals: a removable side cap 110; a body 112; a manual pump mechanism 114; a removable/replaceable a neck/head assembly 116; a removable brush cover 118; and a refillable container or reservoir 120.

Body 112 includes a grip portion 112a and frame portion 112b defining an interior space for accommodating reservoir 120 configured to be filled with and contain an oral health dental product. Neck/head assembly 116 includes an arm portion 116a extending from a top end 112c of body 112 and terminating in a brush portion or head 116b provided with bristles 116c. In this embodiment, arm portion 116a is formed in one piece with brush portion 116b so that the entire neck/head assembly 116 is interchangeable as described above for neck/head assembly 19 in the embodiment of FIGS. 1-8.

Reservoir 120 is contained within body 112 which is provided with an opening at rear side surface thereof. Side cap 110 is configured for removable connection to and disconnection from this opening to permit an oral health dental product to be filled into reservoir 120 as well as to be removed therefrom as desired.

As best shown in FIGS. 11 and 16, a pipe or tube 122 extends into and from reservoir 120 and is connected to a pump element 126 of manual pump mechanism 114. An attachment nozzle 128 with an exterior conical surface is integrally connected to body 112 and extends upwardly from top end 112c of body 112. A lower end of nozzle 128 receives an end of tube 122 and is inserted into a conduit 116e extending along and inside arm portion 116a and terminating at an orifice 116d formed in brush portion 116b. By this construction, nozzle 128, and thus neck/head assembly 116, is in fluid communication with pump mechanism 114 (including pump element 126) and the interior of reservoir 120. Conduit 116e of arm portion 116a is configured to receive and be removably attached to nozzle 128 via pressure fit by means of the exterior conical surface of nozzle 128 engaging a corresponding generally conically-shaped inner portion of conduit 116e. The exterior conical surface of nozzle 128 is provided with a groove 128a configured to receive an O-ring 130 so that a fluid-tight connection is achieved between arm portion 116a and nozzle 128.

Pump element 126 of manual pump mechanism 114 is an activating member in the form of a push-button by means of which a pressure or suction effect is obtained in order to transfer the oral health dental product from reservoir 120 to brush portion 116b of neck/head assembly 116. In the assembled state of toothbrush 100, push-button 126 is exposed from an aperture 112d of body 112 and enclosed with a cover 132 so as to be accessible to the user for actuating pump mechanism 114. As described above for the embodiment of toothbrush 10 shown in FIGS. 1-8, pump mechanisms for toothbrushes are well-known in the art. Any known pump mechanism which can effectively transfer the oral health dental product from reservoir 120 to brush portion 116b is suitable and can be used for toothbrush 100 without departing from the spirit and scope of the present invention.

As noted above, neck/head assembly 116 can be removed from body 112 by disconnecting arm portion 116a from nozzle 128. Toothbrush 100 according to the present invention is therefore configured for use with various interchangeable neck/head assemblies (i.e., brush heads) to provide the user with a total cleaning experience. Brush portion 116b is formed with an orifice 116d in fluid communication with nozzle 128 via arm portion 116a. A spray nozzle 134 is provided in association with orifice 116d at a location corresponding generally to the root of bristles 116c.

As noted above, toothbrush 100 is an electric toothbrush, as compared to manual toothbrush 10 described above with reference to FIGS. 1-8. To that end, toothbrush 100 further includes a battery compartment 136 for containing a battery or battery pack (not shown) that powers an electronic circuit (not shown) which drives an ultrasonic agitator, generally represented by reference numeral 138 in FIGS. 11 and 16, that transmits high-speed vibrations to the brush portion 116b. Toothbrush 100 is turned ON/OFF by an ON/OFF switch 140 mounted to body 112. Switch 140 is exposed from an aperture 112e of body 112 and enclosed with a cover 142 accessible to the user for turning toothbrush 100 ON/OFF. The foregoing electrical components for toothbrush 100 are described and shown only as a mere representation of one way of effectuating vibration of brush portion 116b. Other specific designs and means for effectuating vibration of brush portion 116b known in the art may be used for toothbrush 100 without limitation and without departing from the spirit and scope of the present invention.

During use, electric toothbrush 100 is first turned ON via switch 140. Then, while brushing his/her teeth, for example, the user actuates manual pump mechanism 114 via push-button 126 (e.g., by pressing push-button 126 downward) so that the oral health dental product is transferred from reservoir 120 to brush portion 116b, at which point spray nozzle 134 is configured to spray the oral health dental product in a controlled manner directly into a user's mouth. Preferably, spray nozzle 134 is configured to spray the oral health dental product directly into the user's mouth to effectively cover major areas inside the user's mouth. According to a feature of the present invention, the electric toothbrush 100 is configured to spray the oral health dental product into the user's mouth in the form of a fine, gentle, dispersed mist. This configuration and use of toothbrush 100 promote and improve good dental hygiene and prevent oral disease by allowing users to clean the teeth properly and to provide therapeutic and freshening dental product applications.

Cap 118 is configured (e.g., preferably congruent with the shape of brush portion 116b) to completely cover bristles 116c of brush portion 116b, as shown in the figures, for substantially preventing bristles 16c from any kind of contamination from external sources, as well as for decontaminating bristles 116c after each use of toothbrush 100 as further described below. Cap 118 includes a vent aperture 118a for air circulation, including for drying bristles 116c after each use. Cap 118 therefore constitutes a hygienic cap for toothbrush 100.

According to the present invention, when brush portion 116b is covered and sealed with cap 118, upon actuation of manual pump mechanism 114 via push-button 126 such that the oral health dental product is transferred from reservoir 120 to brush portion 116b, spray nozzle 134 is configured to spray the oral health dental product in a controlled manner directly onto bristles 116c of toothbrush 100 in order to disinfect (e.g., decontaminate, sanitize) toothbrush bristles 116c. According to a feature of the present invention, the oral health dental product is sprayed onto the bristles in the form of a fine, gentle, dispersed mist. Due to the sealing function of cap 118 relative to brush portion 116b, the oral health dental product remains within cap 118 after being sprayed therein as described above to enhance the disinfecting function. By disinfecting bristles 116c in this manner while brush portion 116b is covered with cap 118, toothbrush 100 according to the present invention has the potential to substantially reduce the chances of re-introducing bacteria to the mouth that may otherwise linger on toothbrush 100, particularly when the user is suffering from an illness.

By the foregoing construction and corresponding functions, toothbrush 100 according to the present invention constitutes a self-sanitizing spray toothbrush having dual operational functionalities, namely, for operation by a user while brushing the teeth to selectively dispense an oral health dental product directly into the user's mouth to promote and improve good dental hygiene, and for operation by a user when brush portion 116b is covered with cap 118 to selectively dispense an oral health dental product directly onto bristles 116c for disinfecting or decontaminating bristles 116c. Similar to toothbrush 10 described above with reference to FIGS. 1-8, self-sanitizing spray toothbrush 100 according to the present invention provides a convenient method for the general improvement of oral hygiene practices, including substantially reducing the risk of cross-infection between a sick family member and those who store their toothbrushes within close proximity of the infected toothbrush.

Another aspect of the present invention provides an oral health system containing one or more of any of the toothbrushes and/or toothbrush components of the invention described above with reference to FIGS. 1-16 and one or more dental products for use therewith. In one exemplary embodiment, the toothbrush components of the system include a preselected number of the removable/replaceable neck/head assemblies (toothbrush heads) as described herein, and the one or more dental products include a medicated and/or non-medicated fluid/liquid including one or a combination of a dental rinse, mouth spray, anti-cavity oral rinse, mouth cavity cleanser, mouth spray, dentifrice, freshener, and antibacterial/antiviral for therapeutic, or oral health-hygiene-cosmetics.

The configuration/shapes of the various components for toothbrushes 10 and 100 are not limited to those illustrated in FIGS. 1-16. It is appreciated that other configurations/shapes for such toothbrush components are suitable so long as the various objects and functionalities of the present invention as described herein are achieved. Furthermore, the various components of the toothbrushes according to the present invention described herein with reference to the drawings can be made of high-quality, durable materials known in the art, including suitable metal, plastic and rubber materials.

In the embodiments of the toothbrushes of the present invention described herein with reference to FIGS. 1-16, the toothbrushes are equipped with a manual pump mechanism for feeding the oral health dental product from the reservoir to the brush head. However, it is understood that any of the toothbrushes according to the present invention can be fitted with an electric or electromechanical (rather than manual) pump mechanism without departing from the spirit and scope of the invention.

The previous description of the disclosure is provided to enable any person skilled in the art to make or use the disclosure. Various modifications to the disclosure will be readily apparent to those skilled in the art, and the generic principles defined herein may be applied to other variations without departing from the scope of the disclosure. Thus, the disclosure is not intended to be limited to the examples and designs described herein but are to be accorded the widest scope consistent with the principles and novel features disclosed herein.

What is claimed is:

1. An oral health dental product dispensing toothbrush, comprising:
a brush head;
a spray nozzle mounted in the brush head for spraying an oral health dental product in a controlled manner directly into the user's mouth, the oral health dental product being sprayed in the form of a fine, gentle, dispersed mist;
a body supporting the brush head and having an internal reservoir containing a preselected amount of the oral health dental product and being disposed in fluid communication with the spray nozzle in the brush head; and
a pump mechanism disposed within the body for feeding the oral health dental product from the reservoir to the brush head, wherein upon activation of the pump mechanism the spray nozzle sprays the oral health dental product in a controlled manner and in the form of a fine, gentle, dispersed mist directly into the user's mouth during use of the toothbrush.

2. The oral health dental product dispensing toothbrush of claim 1, wherein upon activation of the pump mechanism, the spray nozzle is further configured to spray an oral health dental product in a controlled manner and in the form of a fine, gentle, dispersed mist directly onto bristles of a brush head in order to decontaminate and/or sanitize the bristles.

3. The oral health dental product dispensing toothbrush of claim 1, wherein the brush head is replaceable.

4. The oral health dental product dispensing toothbrush of claim 1, wherein the oral health dental product is a medicated or non-medicated fluid including one or a combination of a dental rinse, mouth spray, anti-cavity oral rinse, mouth cavity cleanser, mouth spray, dentifrice, freshener, and antibacterial/antiviral for therapeutic or oral health-hygiene-cosmetics.

5. An oral health system comprising: one or more oral health dental product dispensing toothbrushes as in claim 1; and one or more oral health dental products for use with the one or more toothbrushes.

6. The oral health system of claim 5, wherein the oral health dental product is a medicated or non-medicated fluid including one or a combination of a dental rinse, mouth spray, anti-cavity oral rinse, mouth cavity cleanser, mouth spray, dentifrice, freshener, and antibacterial/antiviral for therapeutic or oral health-hygiene-cosmetics.

7. An oral health dental product dispensing toothbrush, comprising:
a brush head having bristles and including a spray nozzle;
a cap configured for covering the brush head and spray nozzle in a substantial sealed state;
a body having an internal reservoir for containing a preselected amount of an oral health dental product and disposed in fluid communication with the spray nozzle in the brush head; and
a pump mechanism for feeding the oral health dental product from the reservoir to the spray nozzle;
wherein upon activation of the pump mechanism during use of the toothbrush to brush the user's teeth, the spray nozzle sprays the oral health dental product in a controlled manner and in the form of a fine, gentle, dispersed mist directly into a user's mouth; and
wherein upon activation of the pump mechanism when the brush head and spray nozzle are covered with the cap, the spray nozzle sprays the oral health dental product in a controlled manner and in the form of a fine, gentle, dispersed mist directly onto the bristles of the brush head to disinfect and/or sanitize the bristles.

8. The oral health dental product dispensing toothbrush of claim 7, wherein the brush head is substantially sealed by the cap such that the oral health dental product remains inside the cap after being sprayed by the spray nozzle to enhance the disinfecting and/or sanitizing function.

9. The oral health dental product dispensing toothbrush of claim 6, wherein the brush head is replaceable.

10. The oral health dental product dispensing toothbrush of claim 7, wherein the oral health dental product is a medicated or non-medicated fluid including one or a combination of a dental rinse, mouth spray, anti-cavity oral rinse, mouth cavity cleanser, mouth spray, dentifrice, freshener, and antibacterial/antiviral for therapeutic or oral health-hygiene-cosmetics.

11. An oral health system comprising: one or more oral health dental product dispensing toothbrushes as in claim 7; and one or more oral health dental products for use with the one or more oral health dental product dispensing toothbrushes.

12. A self-sanitizing toothbrush comprising:
a brush head having bristles and including a nozzle;
a cap configured for covering the brush head and nozzle in a substantial sealed state;
a body having an internal reservoir for containing a preselected amount of an oral health dental product and disposed in fluid communication with the nozzle; and
a pump mechanism for feeding the oral health dental product from the reservoir to the nozzle;
wherein the self-sanitizing spray toothbrush has dual functionalities configured for operation by a user while brushing the teeth to selectively dispense via the nozzle the oral health dental product directly into the user's mouth to promote and improve good dental hygiene, and for operation by the user when the brush head is covered with the cap to selectively dispense via the nozzle the oral health dental product directly onto bristles of the brush head for disinfecting and/or sanitizing the bristles, nozzle being configured to dispense the oral health dental product in a controlled manner and in the form of a fine, gentle, dispersed mist.

13. The self-sanitizing toothbrush of claim 12, wherein the brush head is replaceable.

14. The self-sanitizing toothbrush of claim 12, wherein the oral health dental product is a medicated or non-medicated fluid including one or a combination of a dental rinse, mouth spray, anti-cavity oral rinse, mouth cavity cleanser, mouth spray, dentifrice, freshener, and antibacterial/antiviral for therapeutic or oral health-hygiene-cosmetics.

15. An oral health system comprising: one or more self-sanitizing toothbrushes of claim 12; and one or more oral health dental products for use with the one or more self-sanitizing toothbrushes.

16. The oral health system of claim 15, wherein the oral health dental product is a medicated or non-medicated fluid including one or a combination of a dental rinse, mouth spray, anti-cavity oral rinse, mouth cavity cleanser, mouth spray, dentifrice, freshener, and antibacterial/antiviral for therapeutic or oral health-hygiene-cosmetics.
